(19) United States
(12) Patent Application Publication
(10) Pub. No.: US 2004/0005283 A1
(43) Pub. Date: Jan. 8, 2004

(54) Highly viscous cosmetic product

(76) Inventors: Domnica Cernasov, Ringwood, NJ (US); Brian Kolman, Hillsborough, NJ (US); Alberto Guerrero, Flanders, NJ (US); Steve Attle, San Diego, CA (US); Ralph Macchio, Sparta, NJ (US)

Correspondence Address: Stephan A Pendorf, Pendorf & Cutliff, P O Box 20445, Tampa, FL, 33622-0445, US

(21) Appl. No.: 10/363,109

(22) PCT Filed: Aug. 30, 2001
(86) PCT No.: PCT/EP01/10017

(30) Foreign Application Priority Data
Aug. 31, 2000 (DE) 100 44 062.2

(51) Int. Cl. A61K 7/021

(52) U.S. Cl. 424/63

(57) ABSTRACT

The invention relates to a highly viscous cosmetic product having a novel wax and gel based structure. The inventive cosmetic product is a combination of the structure of a gel, an emulsion, and a wax, and contains the following components (in wt. %): 0.01-8% of a gel-forming substance, between 0.01-3% of the gel-forming substance being gellan gum; 0.01-10% of humectant; 0.1-2% of a hardening agent for the structure of the gel, containing inorganic salts which are at least partially soluble in water; 0.1-10% of a natural or synthetic cosmetic wax; 10-90% of an emulsion-forming agent selected from cosmetic oils and esters, and mixtures of oils, esters and water; 001-15% of an anionic or nonionic emulsifying agent; 4-30 wt. % of pigments. Said product has a penetratin value between 15 and 66, measured by means of a 12 g cone penetrometer.

### BACKGROUND OF THE INVENTION

1. Field of the Invention

The present invention is related to a wax- and water-based highly viscous cosmetic product having a novel structure.

2. Description of the Related Art

From EP-A-976382, cosmetic or pharmaceutic plasters are known which comprise an aqueous phase and contain in a hydrophile gel system the Gellan Gum gel in addition to a hydrocolloid.

### SUMMARY OF THE INVENTION

It is an object of the present invention to provide a novel cosmetic product having a structure hitherto unknown which comprises waxes and gels.

### DETAILED DESCRIPTION OF THE INVENTION

According to the present invention, a cosmetic product is provided which represents a combination of the structure of a gel, an emulsion, and a wax, and which comprises the following components:

0.01-8 weight % of one or several gel formers wherein the share of the Gellan gum gel former ranges between 0.01-3 weight %,

0.01-10 weight % of a moisturizer,

0.1-5 weight % of a hardening agent for the structure of the gel comprising inorganic salts which are at least partly soluble in water,

0.1-10 weight % of natural or synthetic cosmetic waxes or mixtures thereof,

10-90 weight % of an emulsion former selected among cosmetic oils, esters, mixtures of oils and esters, and water,

0.01-15 weight % of an anionic or non-ionic emulsifier or a mixture thereof, and

4-30 weight % of a pigment or mixture of pigments,

wherein the product has a Penetration value ranging from 15 to 66, measured by a penetrometer, 12 gr. cone, and wherein all percents are related to the entire weight of the product.

Due to its structure, the novel cosmetic product is manufactured as a pigmented product which comprises preferably colored pigments such as iron oxides. While in common gels according to the prior art such as the gel disclosed in EP-A-976382 pigments like iron oxide break the gel structure, the cosmetic product according to the present invention allows without any problem the inclusion of common pigments in concentrations up to 30 weight %. The preferred range is 4-24 weight %, more preferred 5 to 15 weight % of pigments. These pigments include iron oxides, natural aluminium silicates such as ochre, titanium dioxide, mica, kaolin, manganese-containing clays such as umbra and red bolus, calcium carbonate, talc, silica, mica-titanium oxide, mica-titanium oxide-iron oxide, mica-titanium oxide-organic dye, bismuth oxychloride, nylon beads, ceramic beads, expanded and non-expanded synthetic polymer powders, powdery natural organic compounds such as minced tight algae, minced plant parts, encapsulated and non-encapsulated cereal starches and mixtures thereof.

Further, the gel according to the present invention has a very high Penetration value (hardness). From practical reasons it is better to work with the Penetration value than with the viscosity. Preferred ranges of the Penetration value are for thin sticks (e.g. 0-15 mm) such as lipsticks, fat sticks, make-up sticks 15 to 30, for thicker sticks (e.g. >15 mm) such as foundation sticks 31 to 66 and for compacts 42 to 52.

The measurement of the penetration value takes place with a penetrometer, e.g. Lab-Line, manufactured by Lab-Line Instruments Inc., USA, cone 12 gr., weights 50 gr. A ring with a height of 3.5 cm, an outer diameter of 3.7 cm and an inner diameter 3.4 cm is placed onto a piece of paraffin paper. A sample is heated to its pouring temperature, poured in the ring and allowed to cool for 12-24 hours in a controlled environment at 25° C. After that the paraffin paper is peeled off from the ring and measurements be taken from this side of the ring.

The cone is mounted in the penetrometer head, the weight is added to the loading bar and the measuring head is lowered so that the tip of the cone touches the surface of the sample. After that the plunger is released gently and the cone let be penetrated the sample for 5 sec. Than the measured value is read. Four values are taken from different places of the sample and reported as an average.

The hardening agent for the structure of the gel in the composition comprises preferably inorganic salts which are selected from the group consisting of sodium, magnesium, potassium, calcium, manganous(II), barium, ferrous, ferric salts and mixtures thereof.

Further, the cosmetic product according to the present invention comprises natural or synthetic waxes or mixtures thereof, preferably in a range between 0.1 and 10 weight %. Suitable waxes may include natural vegetable waxes, animal waxes, natural and synthetic mineral waxes and synthetic waxes. These include for example, carnauba wax, candelilla wax, beeswax, wool wax, paraffin, ceresin, ozokerite, silicon, polyethylene glycol or glycol ester waxes, montan wax, and mixtures thereof.

The gel former Gellan gum used in the present invention is a polysaccharide produced by controlled fermentation of the organism Pseudomonas eloeda. Gellan gum has been used as a multi-functional hydrocolloid especially in the food industry. Gelation occurs by cations such as sodium, potassium, magnesium, calcium and hydrogen ions which cations typically found or added in many cosmetic products. The ion concentration has an influence to the strength of the gel and can be adapted by the skilled in the art. A preferred Gellan gum is Kelcogel® of NutraSweet, San Diego, 8355 Aero Drive, San Diego, Calif. 92123-1718, USA.

Further gel formers may be present in combination with Gellan gum in the cosmetic product according to the present invention, such as carbomer, xanthane rubber, carrageenan, acacine, guar rubber, agar-agar, alginates and tyloses, carboxymethylcellulose, hydroxyethylcellulose, certain polyacrylates, polyvinyl alcohol, polyvinyl pyrrolidone, montmorillonite, and mixtures thereof. Here, the proportion of Gellan gum ranges between 0.01-3 weight %, and another gel former like, preferably, xanthan rubber, may be present in the range of 0.01—about 8 weight %. A combination of Gellan gum with one or more other gel formers is advantageous because it helps to balance the gel hardness.

The emulsion is the third main component apart from the wax and the gel. It is a solid (granulate or pastic) emulsion it is e.g. built with a common cosmetic oil or an ester or an ether, and forms together with water an O/W or W/O emulsion.

Suitable oils and esters include for example mineral oil, hydrogenated polyisobutane, synthetic squalane or squalane made of natural products; cosmetic esters or ethers which may be branched or unbranched, saturated or unsaturated; vegetable oils; or mixtures of two ore more thereof.

Particularly suitable oils are for instance mineral oils, hydrogenated polyisobutane, polyisoprene, squalane, tridecyltrimellitate, trimethylpropane-triisostearate, isodecylcitrate, neopentylglycol-diheptanoate, PPG-15-stearylether as well as vegetable oils such as calendula oil, jojoba oil, avocado oil, macadamia nut oil, castor oil, wheat germ oil, vine kernel oil, kukui nut oil, thistle oil, night candle oil, saflor oil, or a mixture of several thereof.

Suitable esters or ethers include for example (INCI denominations): dipentaerythrityl hexacaprilate/hexacaprate/tridecyl trimellitate/tridecyl stearate/neopentyl glycol dicaprylate dicaprate, propylene glycol dioctanoate 5, propylene glycol dicaprylate 2,30 dicaprate, tridecyl stearate/neopentyl glycol dicaprylate dicaprate/tridecyl trimellitate, neopentyl glycol dioctanoate, isopropyl myristate, diisopropyl dimer dilinoleate, trimethylpropane triisostearate, myristyl ether, stearyl ether, butyl ether, dicaprylyl ether, PPG-1-PEG9 lauroyl glycol ether, PPG15 stearyl ether, PPG14 butyl ether, Fomblin HC25.

Suitable emulsifiers include addition products of 2-30 mol ethylene oxide to linear C₈-C₂₂ fatty alcohols, to C₁₂-C₂₂ fatty acids, and to C₈-C₁₅ alkylphenoles; C₁₂-C₂₂ fatty acid mono and diesters of addition products of 1-30 mol ethylene oxide to glycerol; or addition products of 2-15 mol ethylene oxide to castor oil; esters of C₁₂-C₂₂ fatty acids and glycerol, polyglycerol, pentaerythrite, sugar alcohols (e.g. sorbite), polyglucosides (e.g. cellulose); polyalkylene glycols; wool wax alcohols; copolymers of polysiloxane-polyalkyl polyether; lecithin. Preferred products are for instance Tween 20 or Span 60.

Necessary for the structure of the product are hardening agents such as for example Behenyl Erucate or waxes such as montan wax, carnauba wax, ozokerite, candelilla wax and mixtures thereof in the range of 0.1-10 weight %.

Suitable moisturizers are for example glycerol, butylene glycol, propylene glycol, algae extracts, NaPCA, Aloe vera, allantoin, panthenol.

The cosmetic product according to the present invention may comprise further agents such as inorganic and organic light protecting agents, radical scavenger, vitamins, enzymes, vegetable agents, algae extracts, polymers, melanin, antioxidants, natural anti-inflammatory agents. It may further comprise common auxiliary substances such as propylene glycol, lecithin, caprylic/capric triglyceride, diisopropyl dimer dilinolate, neopentyl glycol dicaprate, sorbitane stearate, acrylates/carbamate copolymer, cations, saccharide isomerate, preservatives, perfumes, etc.

The cosmetic product according to the present invention has a specific texture because it can be in appearance with a pressed powder with the application of a liquid and the aspect of a “hot poured” product.

It may be used for instance as free standing stick, e.g. for lipsticks, lip gloss, lip balm; as pourable substance for various containers, for example for greasepaint, primer substances, make-up, as patches for local treatment of certain skin areas; or as crayons.

The cosmetic product according to the present invention is manufactured in that at first the gel is formed by adding the gel former to water at a temperature ranging between 75 and 80° C. The oil phase manufactured separately which comprises waxes and esters plus further components such as moisturizers, further hardening agents, emulsifiers and certain oils, is then added at 75 to 80° C. while stirring.

Finally, the mixture is homogenized at that temperature and cast into molds or pans after cooling down to 65 to 80° C.

Now, the present invention will be further explained by way of examples. All percents are given as weight percent provided that nothing else is specified.

EXAMPLE 1
Cool lipstick

| | % |
|---|---|
| Phase A | |
| Water, deionized | to 100 |
| Gellan gum | 0.8 |
| Butyl englycol | 2 |
| Xantham gum | 0.3 |
| Phase B | |
| Polysorbate | 2 |
| Phase C | |
| Glycerin | 11 |
| Colour pigments | 6 |
| Silica | 1 |
| Nylon-12 | 3 |
| Talc | 6 |
| Phase D | |
| Candelilla wax | 4.5 |
| Ozokerite | 0.5 |
| Lecithin | 1.5 |
| Preservatives | 0.5 |
| Behenyl Erucate | 0.7 |
| Diisopropyl Dimer Dilinoleate | 2 |
| Neopentyl Glycol Dicaprate | 2 |
| Sorbitan Stearate | 2 |

The gel formers were stirred with water and heated with the other components of phase A to about 75° C. The separate mixed phases B, C and D were added to phase A at that temperature while stirring. The mixture was homogenized at about 75° C., cooled down to 70° C. and cast into lipstick molds. The lipstick had a penetration value of 21.

EXAMPLE 2
Cool foundation stick

| Phase A | |
|---|---|
| Water | to 100 |
| Gellan gum | 0.5 |
| Butylenglycol | 2 |
| Xantham gum | 0.3 |
| Sodium chloride | 0.2 |
| Phase B | |
| Polysorbate | 2 |
| Phase C | |
| Glycerin | 11 |
| Color pigments | 8.5 |
| Silica | 1.5 |
| Boron nitride | 2 |
| Talc | 4 |
| Phase D | |
| Sorbitan Stearate | 2.5 |
| Candelilla wax | 4.5 |
| Ozokerite | 0.8 |
| Ceresin wax | 1.5 |
| Lecithin | 1 |
| Preservatives | 0.5 |
| Caprylic Capric Triglyceride | 3.5 |
| Diisopropyl Dimer Dilinoleate | 2 |
| Neopentyl Glycol Dicaprate | 3 |

The manufacturing was according to example 1. The foundation stick had a penetration value of 55.

EXAMPLE 3
Compact shad

| Phase A | |
|---|---|
| Water | to 100 |
| Gellan gum | 0.7 |
| Butylenglycol | 3 |
| Xantham gum | 0.3 |
| Allantoin | 0.01 |
| Phase B | |
| Polysorbate | 2 |
| Phase C | |
| Glycerin | 10 |
| Color pigments | 6 |
| Silica | 1 |
| Barium sulphate | 2 |
| Talc | 5 |
| Nylon-12 | 3 |
| Phase D | |
| Candelilla wax | 3.5 |
| Ozokerite | 0.5 |
| Lecithin | 1.5 |
| Behenyl Erucate | 0.5 |
| Caprylic Capric Triglyceride | 3 |
| Diisopropyl Dimer Dilinoleate | 2 |
| Neopentyl Glycol Dicaprate | 3 |
| Sorbitan Stearate | 2.5 |
| Phase E | |
| Algae extract/Tocopherol/Retinyl palmitate | 0.1 |
| Preservatives | 0.2 |
| Fragrance | 0.3 |

The manufacturing was according to example 1. The compact foundation had a penetration value of 47.

1. Highly viscous cosmetic product which comprises the combination of the structure of a gel, an emulsion, and a wax, wherein said combination comprises
0.01-8 weight % of one or several gel formers wherein the share of the Gellan gum gel former ranges between 0.01-3 weight percent,
0.01-10 weight % of a moisturizer,
0.1-5 weight % of a hardening agent for the structure of the gel comprising inorganic salts which are at least partly soluble in water,
0.1-10 weight % of natural or synthetic cosmetic waxes or mixtures thereof,
10-90 weight % of an emulsion former selected among cosmetic oils, esters, mixtures of oils and esters, and water,
0.01-15 weight % of an anionic or non-ionic emulsifier or a mixture thereof, and
4-30 weight % of a pigment or mixture of pigments,
wherein the product has a Penetration value ranging from 15 to 66, measured by a penetrometer, 12 gr. cone, and wherein all percents are related to the entire weight of the product.

2. Cosmetic product according to claim 1, which comprises 4 to 24 weight % of pigments.

3. Cosmetic product according to claim 2, which comprises 5 to 15 weight % of pigments.

4. Cosmetic product according to claim 1, which comprises as pigments iron oxides, natural aluminium silicates, titanium oxide, mica, kaolin, manganese-containing clays, talc, nylon beads, silicon dioxide, and mixtures thereof.

5. Cosmetic product according to claim 1, wherein the share of the natural or synthetic waxes or mixtures thereof ranges between 0.1-10 weight %.

6. Cosmetic product according to claim 1, wherein the waxes are selected among paraffin wax, carnauba wax, montan wax, candelilla wax, ozokerite, and mixtures thereof.

7. Cosmetic product according to claim 1, wherein apart from Gellan gum, further gel formers of the group are present, being selected among carbomer, xanthan rubber, carrageenan, acacine, guar rubber, agar-agar, alginates, PVA, PVP, and mixtures thereof.

8. Cosmetic product according to claim 1, wherein the inorganic salts are selected from the group consisting of sodium, magnesium, potassium, calcium, manganous(II), barium, ferrous, ferric salts and mixtures thereof.

9. Cosmetic product according to claim 1, wherein the product is a stick of 5-15 mm diameter with a penetration value of 15 to 30.

10. Cosmetic product according to claim 1, wherein the product is a stick of >15 mm diameter with a penetration value of 31 to 6.

11. Cosmetic product according to claim 1, wherein the product is a compact foundation with a penetration value of 42 to 52.
